(19) United States
(12) Patent Application Publication
(10) Pub. No.: US 2024/0252171 A1
(43) Pub. Date: Aug. 1, 2024

(54) SURGICAL INSTRUMENTS HAVING A REINFORCED STAPLE CARTRIDGE

(71) Applicant: Intuitive Surgical Operations, Inc., Sunnyvale, CA (US)

(72) Inventors: Matthew Wixey, San Jose, CA (US); Babak Jasemian, Trabuco Canyon, CA (US)

(73) Assignee: Intuitive Surgical Operations, Inc., Sunnyvale, CA (US)

(21) Appl. No.: 18/601,714

(22) Filed: Mar. 11, 2024

Related U.S. Application Data
(63) Continuation of application No. 17/414,780, filed on Jun. 16, 2021, now Pat. No. 11,944,301.
(60) Provisional application No. 62/783,444, filed on Dec. 21, 2018.

(51) Int. Cl. A61B 17/072; A61B 17/00; A61B 17/068; A61B 34/37

(52) U.S. Cl. CPC A61B 17/07207; A61B 17/068; A61B 17/072; A61B 34/37; A61B 2017/00314; A61B 2017/07271; A61B 2017/07278; A61B 2017/07285

(57) ABSTRACT

The present disclosure provides a surgical instrument, such as a tissue sealing instrument, with an elongate shaft and first and second jaws movably coupled to the shaft. The second jaw comprises a cavity for receiving a staple cartridge and a substantially longitudinal reinforcing wall extending through the cavity. The instrument further includes a drive member configured to translate through the end effector to close the jaws and to engage a plurality of staples within the staple cartridge to drive the staples into tissue. The reinforcing wall provides additional support for the staple cartridge to inhibit deformation of the staple cartridge during actuation, thereby improving the staple formation in the tissue. This improves the tissue seal provided by the staples without increasing the overall size of the end effector.

### CROSS-REFERENCE TO RELATED APPLICATIONS

This application is a continuation application of U.S. application Ser. No. 17/414,780 filed Jun. 16, 2021, which is the National Stage of International Application No. PCT/US2019/062768 filed Nov. 22, 2019, which claims the benefit of U.S. Provisional Application No. 62/783,444, filed Dec. 21, 2018, the entire disclosures of each are incorporated herein by reference for all purposes.

### BACKGROUND

The field of the present disclosure relates to medical instruments, and more particularly to tissue sealing instruments for use in surgeries. Even more particularly, the present disclosure relates to a surgical stapling instrument having a reinforced staple cartridge and a novel drive assembly.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Minimally invasive telesurgical robotic systems are being developed to increase a surgeon's dexterity when working on an internal surgical site, as well as to allow a surgeon to operate on a patient from a remote location (outside the sterile field). In a telesurgery system, the surgeon is often provided with an image of the surgical site at a control console. While viewing a three dimensional image of the surgical site on a suitable viewer or display, the surgeon performs the surgical procedures on the patient by manipulating master input or control devices of the control console, which in turn control motion of the servo-mechanically operated slave instruments.

The servomechanism used for telesurgery will often accept input from two master controllers (one for each of the surgeon's hands) and may include two or more robotic arms. A surgical instrument is mounted on each of the robotic arms. Operative communication between master controllers and associated robotic arm and instrument assemblies is typically achieved through a control system. The control system typically includes at least one processor that relays input commands from the master controllers to the associated robotic arm and instrument assemblies and back in the case of, for example, force feedback or the like. One example of a robotic surgical system is the DA VINCI™ system commercialized by Intuitive Surgical, Inc. of Sunnyvale, Calif.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. Nos. 7,594,912, 6,758,843, 6,246,200, and 5,800,423, the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. Nos. 6,702,805, 6,676,669, 5,855,583, 5,808,665, 5,445,166, and 5,184,601, the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. For this reason, it is desirable to provide surgical tools that include mechanisms that provide two or three degrees of rotational movement of an end effector to mimic the natural action of a surgeon's wrist. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions. Surgical instruments are often deployed into restrictive body cavities (e.g., through a cannula to inside the pelvis). Accordingly, it is desirable for the surgical instrument to be both compact and maneuverable for best access to and visibility of the surgical site.

Conventional surgical clamping and cutting instruments typically include an end effector with a fixed jaw and a movable jaw that can be opened and closed relative to the fixed jaw. A staple cartridge is often designed to fit within one of the jaws of the end effector. The staple cartridge contains multiple rows of staple assemblies that each includes a staple and a staple driver, sometimes referred to as a staple pusher. The staple pusher holds the staple in place prior to use, and then drives the staple into tissue when the instrument is actuated. A drive member is advanced through the staple cartridge to close the jaws and engage the staples, thereby driving the staples into tissue. An actuator, such as a cable or coil, advances the drive member distally through an internal channel in one of the jaws of the end effector. Prior to actuation of the staples, the jaws are in a “closed” position around the tissue. In this closed position, the jaws are typically parallel to each other such that the distance between the upper and lower jaws is a fixed dimension along the length of the jaws. This parallel tissue gap between the jaws places the jaws in close cooperative alignment for clamping, sealing and/or holding the tissue in place prior to stapling.

In many known surgical instruments, the staple cartridge is a separate component that is loaded into one of the jaws of the end effector. For cost and ease of manufacturing reasons, the staple cartridge is typically made of a polymer material and, therefore, is not as sturdy as the surrounding metal jaws on the end effector. One of the drawbacks with these designs is that the polymer staple cartridge may deform slightly as the drive member advances through the end effector and drives the staples into tissue, particularly if the tissue to be clamped is relatively thick (e.g., 2-5 mm). Deformation of the staple cartridge can cause deformation or misdirection of the staples as they are driven into the tissue, resulting in an imperfect staple formation within the tissue. This reduces the overall clamping and sealing ability of the device.

To improve the sealing ability of conventional staple cartridges, multiple rows of staples have been used. For example, many known surgical clamping instruments utilize three or more rows of staples at least in part to inhibit deformation of the cartridge and provide an effective seal of tissue during a procedure. While increasing the number of staple rows has improved the tissue seal provided by these devices, it has also increased the overall cross-sectional size of these devices, which is in direct contrast to the growing requirements for smaller and more maneuverable instruments in surgical procedures.

Accordingly, while the new telesurgical systems and devices have proven highly effective and advantageous, still further improvements would be desirable. In general, it would be desirable to provide improved surgical instruments that are more compact and maneuverable to enhance the efficiency and ease of use of minimally invasive systems. More specifically, it would be beneficial to provide improved surgical clamping and sealing instruments that reduce deformation of the staple cartridge during use to improve the tissue seal, while maintaining or reducing the overall size of the devices.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

The present disclosure relates to a surgical instrument comprising an end effector with first and second jaws configured to move relative to each other from an open position to a closed position. The second jaw comprises a cavity for receiving a staple cartridge and a substantially longitudinal reinforcing wall extending through the cavity of the second jaw. The instrument further includes a drive member configured to translate through the end effector to close the jaws and to engage a plurality of staples within the staple cartridge to drive the staples into tissue. The reinforcing wall provides additional support for the staple cartridge once it has been loaded into the second jaw to inhibit deformation of the staple cartridge during actuation, thereby improving the staple formation in the tissue. This improves the tissue seal provided by the staples without increasing the size of the end effector.

In an exemplary embodiment, at least a portion of the reinforcing wall comprises a sturdy material, such as metal or the like. This design allows for the use of a polymer staple cartridge, e.g., plastic, as the metal reinforcing wall provides central support along substantially the entire length of the staple cartridge to inhibit or prevent deformation of the cartridge during use.

In certain embodiments, the surgical instrument further includes a staple cartridge having first and second elongate side portions and the reinforcing wall defines first and second longitudinal grooves within the cavity of the second jaw for receiving the side portions of the staple cartridge. The grooves have lateral walls on both sides of the side portions of the staple cartridge to provide additional support for the cartridge during actuation. Thus, the two side portions of the staple cartridge are substantially surrounded by the metallic walls of the jaw (i.e., outer lateral walls and inner reinforcing wall), which reinforces the staple cartridge to inhibit deformation during use.

In an exemplary embodiment, the reinforcing wall comprises first and second lateral walls defining a channel therebetween for receiving a portion of the drive member therethrough. The reinforcing wall may for example, comprise a slotted spline that provides lateral wall support for each side portion of the stapler cartridge while allowing for passage of a portion of the drive member through the central slot or channel in the spline.

In certain embodiments, the staple cartridge will comprise first and second rows of staple assemblies that each include one or more staples and staple pushers. The reinforcing wall of the second jaw allows for the design of a staple cartridge with only two rows of staple assemblies (in contrast to conventional surgical instruments that often include three or more rows), while still providing sufficient support to the staple cartridge to inhibit or prevent deformation of the cartridge during actuation. Providing only two rows of staple assemblies within the cartridge allows for the design of a more compact and maneuverable surgical instrument.

In certain embodiments, the staple cartridge is designed to “drop down” into the cavity of the second jaw, i.e., from a direction transverse, preferably perpendicular, to the longitudinal axis of the second jaw. In these embodiments, the second jaw may include outer walls, an internal reinforcing wall and distal and proximal walls that all form a cavity with two longitudinal grooves for receiving the first and second side portions of the staple cartridge. The cartridge is preferably loaded “vertically” into this cavity to provide optimal structural support for the stable cartridge within the second jaw, thereby inhibiting deformation of the cartridge during actuation.

In a preferred embodiment, the drive member comprises one or more inclined ramps configured to engage the staples and drive the staples in a direction transverse to the longitudinal axis as the drive member is translated through the housing, The inclined ramps are preferably an integral part of the drive member (rather than as part of the second jaw or the staple cartridge), which provides more space in the staple cartridge and the second jaw for reinforcing the staple cartridge.

In an exemplary embodiment, the drive member includes a first portion that translates through a channel in the first jaw, and an actuation mechanism in contact with the first portion of the drive member. The actuation mechanism is configured to translate the drive member distally through the end effector. The actuation mechanism advances the drive member through the first jaw (instead of through an internal channel in the staple cartridge as in conventional surgical stapling instruments). Eliminating the internal channel for the actuation mechanism from the staple cartridge provides additional space for the reinforcing wall in the second jaw.

In a preferred embodiment, the actuation mechanism includes a cable or coil that applies a distal force to the first portion of the drive member. The coil is configured to pass through at least a portion of the channel in the first jaw to translate the drive member distally through the end effector. In certain embodiments, an actuator is operatively coupled to the actuation mechanism. In a preferred embodiment, the actuator includes a control device of a robotic surgical system.

In embodiments, the end effector includes a pair of jaws configured to grasp tissue. In embodiments, the end effector includes a pair of jaws configured to staple and cut tissue.

In another aspect of the invention, a staple cartridge in accordance with this disclosure includes an elongate base configured for placement into a cavity of a jaw of a surgical stapling instrument The cartridge further includes first and second side portions extending from the base in a direction substantially perpendicular to the longitudinal axis of the base and defining a substantially longitudinal channel therebetween. The first and second side portions each include at least one row of staple retention openings and are configured to fit within longitudinal grooves in the cavity of the surgical instrument jaw. The first and second side portions allows the staple cartridge to be supported by a central reinforcing wall in the jaw, which reduces deformation of the cartridge during actuation. The staple cartridge is preferably made at least in part of a polymer, such as plastic or other suitable material.

In yet another aspect of the invention, a surgical instrument comprises an end effector with first and second jaws configured to move relative to each other from an open position to a closed position. The second jaw includes a cavity for receiving a staple cartridge containing a plurality of staples. The instrument further includes a drive member configured to translate longitudinally through the end effector to move the first and second jaws from the open position to the closed position. The drive member comprises one or more inclined ramps configured to engage the staples and drive the staples in a direction transverse to a longitudinal axis of the instrument as the drive member is translated longitudinally through the housing, In preferred embodiments, the inclined ramps are integral with the drive member (rather than disposed within the second jaw or the staple cartridge).

In certain embodiments, the drive member is configured for operation by a robotic surgical system. In other embodiments, the drive mechanism is configured for operation by an electro-mechanical powered instrument or a manually powered instrument.

The instrument may include an actuation mechanism in contact with the drive member and configured to translate the drive member distally through the end effector and an actuator operatively coupled to the actuation mechanism. The actuator may include a control device of a robotic surgical system.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The above and other aspects, features, and advantages of the present surgical instruments will become more apparent in light of the following detailed description when taken in conjunction with the accompanying drawings in which:

FIG. 1 is a perspective view of an illustrative surgical instrument in accordance with the present disclosure;

FIG. 1A is a perspective view of the distal end portion of an illustrative surgical instrument in accordance with the present disclosure with the jaws in the open position;

FIG. 1B is a perspective view with parts separated of the end effector of FIG. 1A;

FIG. 1C is a perspective exploded view of a staple cartridge, staples, and staple pushers for use with a surgical instrument according to the present disclosure;

FIG. 2A is a bottom perspective view of a staple cartridge in accordance with the present disclosure;

FIG. 2B is a top perspective view of a cartridge receiving jaw in accordance with the present disclosure;

FIG. 3 is a cross-sectional view of an end effector of an illustrative surgical instrument in accordance with the present disclosure in the closed position;

FIG. 4 is a cross-sectional side of a clevis according to the present disclosure;

FIG. 5 is a perspective view of the end portion of an illustrative surgical instrument with parts removed;

FIG. 6 is a perspective view of a drive member in accordance with the present disclosure;

FIG. 7A is a cross-sectional perspective view of the actuation mechanism for the drive member of FIG. 6;

FIG. 7B is a cross-sectional side view of the actuation mechanism for the drive member of FIG. 6;

FIG. 8A shows a movable lower jaw of an illustrative surgical instrument in an open configuration;

FIG. 8B shows a movable lower jaw of an illustrative surgical instrument pivoting towards a closed position;

FIG. 8C shows a movable lower jaw of an illustrative surgical instrument in a closed position;

FIG. 9 illustrates a top view of an operating room employing a robotic surgical system utilizing aspects of the present invention; and

FIG. 10 illustrates a simplified side view of a robotic arm assembly that is usable with various aspects of the present invention.

### DETAILED DESCRIPTION

Particular embodiments of the present surgical instruments are described hereinbelow with reference to the accompanying drawings; however, it is to be understood that the disclosed embodiments are merely exemplary of the disclosure and may be embodied in various forms. Therefore, specific structural and functional details disclosed herein are not to be interpreted as limiting, but merely as a basis for the claims and as a representative basis for teaching one skilled in the art to variously employ the present disclosure in virtually any appropriately detailed structure. Well-known functions or constructions are not described in detail to avoid obscuring the present disclosure in any unnecessary detail.

While the following disclosure is presented with respect to a linear surgical stapler where staples are sequentially fired, it should be understood that the features of the presently described surgical instruments may be readily adapted for use in any type of surgical grasping, clamping, cutting, or sealing instruments, whether or not the surgical clamping and cutting instrument applies a fastener. For example, the presently described drive member and actuation mechanism may be employed in an electrosurgical instrument wherein the jaws include electrodes for applying energy to tissue to treat (e.g., cauterize, ablate, fuse, or cut) the tissue. The surgical clamping and cutting instrument may be a minimally invasive (e.g., laparoscopic) instrument or an instrument used for open surgery. Additionally, the features of the presently described surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

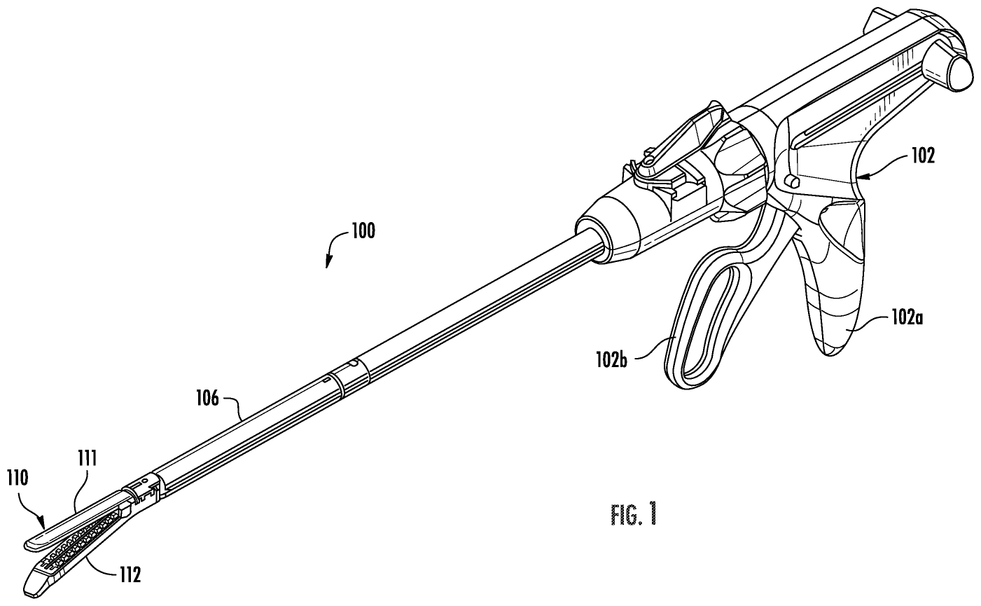

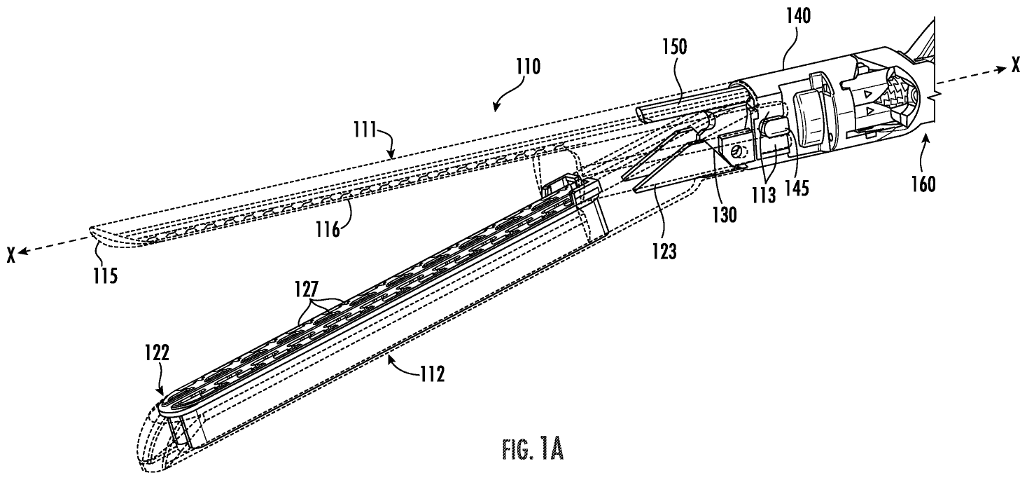

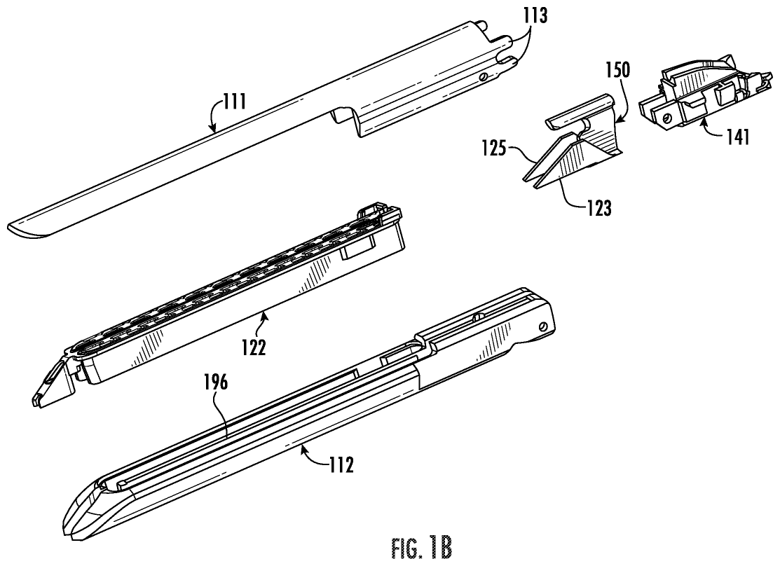

FIG. 1 is a perspective view of a representative surgical stapling instrument 100 in accordance with certain embodiments of the present disclosure having a handle assembly 102, and an end effector 110 mounted on an elongated shaft 106 of the surgical stapling instrument 100. End effector 110 includes a first jaw 111 and a second jaw 112. Handle assembly 102 includes a stationary handle 102a and a moveable handle 102b, which serves as an actuator for surgical instrument 100.

In certain embodiments, handle assembly 102 may include input couplers (not shown) instead of, or in addition to, the stationary and movable handles. The input couplers provide a mechanical coupling between the drive tendons or cables of the instrument and motorized axes of the mechanical interface of a drive system. The input couplers may interface with, and be driven by, corresponding output couplers (not shown) of a telesurgical surgery system, such as the system disclosed in U.S Pub. No. 2014/0183244A1, the entire disclosure of which is incorporated by reference herein. The input couplers are drivingly coupled with one or more input members (not shown) that are disposed within the instrument shaft 106. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. Nos. 8,597,280, 7,048,745, and 10,016,244. Each of these patents is hereby incorporated by reference in its entirety.

Actuation mechanisms of surgical instrument 100 may employ drive cables that are used in conjunction with a system of motors and pulleys. Powered surgical systems, including robotic surgical systems that utilize drive cables connected to a system of motors and pulleys for various functions including opening and closing of jaws, as well as for movement and actuation of end effectors are well known. Further details of known drive cable surgical systems are described, for example, in U.S. Pat. Nos. 7,666,191 and 9,050,119 both of which are hereby incorporated by reference in their entireties for all purposes. While described herein with respect to an instrument configured for use with a robotic surgical system, it should be understood that the wrist assemblies described herein may be incorporated into manually actuated instruments, electro-mechanical powered instruments, or instruments actuated in any other way.

FIG. 1A shows the distal end portion of surgical instrument 100, including an end effector 110 defining a longitudinal axis X-X and having a first jaw 111, a second jaw 112, a clevis 140 for mounting jaws 111, 112 to the instrument, and an articulation mechanism, such as a wrist assembly 160. First jaw 111 includes an anvil 115 having staple-forming pockets 116. In certain embodiments, second jaw 112 is a movable jaw configured to move from an open position to a closed position relative to first jaw 111. In other embodiments, first jaw 111 is a movable jaw configured to move between open and closed positions relative to second jaw 112. In still other embodiments, both jaws 111, 112 are movable relative to each other. In the open position, a fresh stapling cartridge 122 (sometimes referred to as a reload) can be loaded into movable jaw 112 and tissue may be positioned between the jaws 111, 112. In the closed position, jaws 111, 112 cooperate to clamp tissue such that cartridge 122 and the anvil 115 are in close cooperative alignment. A drive member 150 is configured to translate distally and, in some embodiments, retract proximally through the end effector. Drive member 150 includes a shuttle 123 with one or more inclined distal portions or ramps 125 formed thereon.

Referring now to FIGS. 1B and 1C, a representative staple cartridge 122 may include a plurality of staple assemblies, each comprising one or more staples 124 supported on corresponding staple drivers or pushers 126 provided within respective staple apertures 127 formed in cartridge 122. The staple assemblies each include at least one (preferably 2-4) staple pushers 126 removably coupled to at least one (preferably 2-4) staples 124. The staple assemblies are preferably arranged within apertures 127 such that staple pusher 126 is situated near a bottom surface of staple cartridge 122 and staples 124 have their legs facing a top surface of cartridge 122. Staple pushers 126 may be manufactured together as a single piece, or they may be separately molded and then coupled to each other and/or arranged relative to each other and staples 124 within cartridge 122.

As discussed above, the entire staple cartridge 122 can be loaded into a jaw of an end effector for use in surgery as described in more detail below. In certain embodiments, staple pusher(s) 126 include one or more supporting elements extending above their top surface for providing support to staples 124 when they are resting thereon. Of course, other suitable geometric designs of staple pusher 126 may be used to receive and hold staple 124 in accordance with the present invention. For example, pusher 126 may have a recess (not shown) for receiving staple 124, as is described in commonly-assigned, co-pending provisional patent application Ser. No. 62/855,371, filed May 31, 2019. Alternatively, pusher 126 may have a flatter upper surface (i.e., without a recess or pocket) that allows the backspan of staple 124 to rest thereon, as is described in commonly-assigned, co-pending provisional patent application Ser. No. 62,783,460, the complete disclosures of both of these applications are hereby incorporated by reference in their entirety for all purposes.

In certain embodiments of the present disclosure, cartridge 122 also may include a shuttle 123 having an inclined distal surface 125 that, upon distal movement, sequentially acts on staple pushers 126, camming them upwardly, thereby moving staples 124 into deforming contact with an anvil of a surgical instrument. Shuttle 123 may be part of drive member 150 (FIGS. 1B and 6) described in more detail below. Cartridge 122 may be removably received within a jaw of a surgical instrument or, in single use embodiments, may be manufactured as part of the jaw.

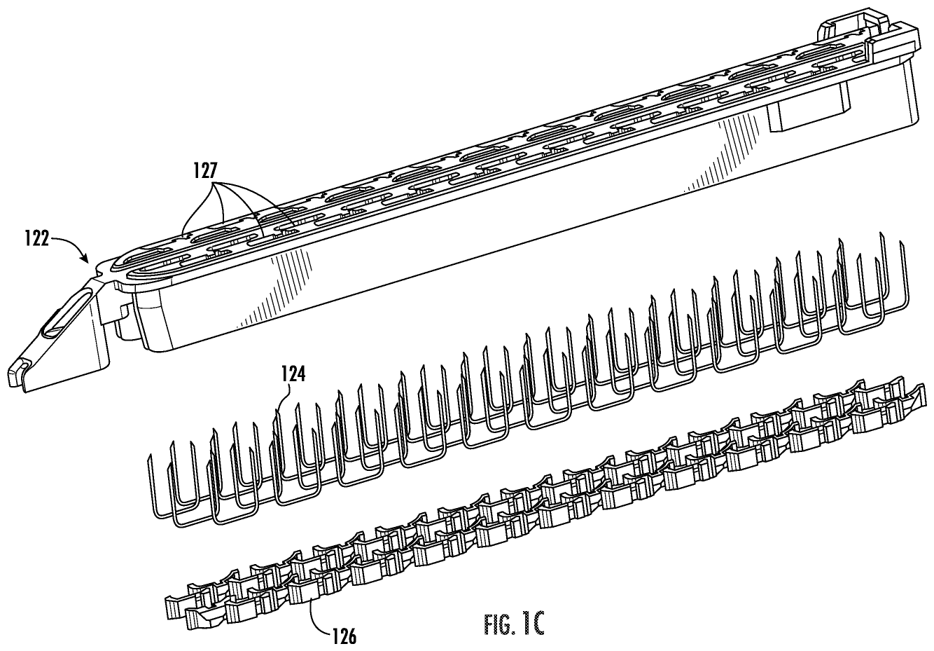

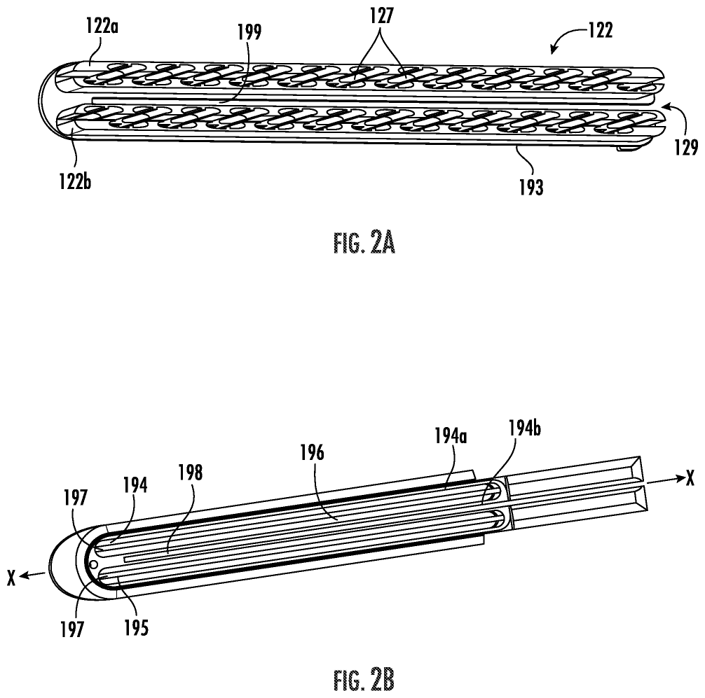

As shown in FIG. 2A, a preferred embodiment of cartridge 122 according to the present disclosure includes a first elongate side portion 122a and a second elongate side portion 122b. A channel 129 is defined between side portions 122a, 122b of cartridge 122. Each side portion 122a, 122b of cartridge 122 includes two rows of staple retention openings 127 configured to receive staples 124 supported on corresponding staple pushers or drivers 126. A slot 199 is located above channel 129 and extends through tissue contacting surface 193 of cartridge 122. Cartridge 122 is preferably formed of a polymer, such as plastic or other suitable material, for cost and ease of manufacturing reasons. Of course, it will be recognized that cartridge 122 may be formed of any material that provides sufficient rigidity to withstand the forces applied to cartridge 122 when drive member 150 engages staple pushers 126 and drives staples 124 into tissue.

As shown in FIG. 2B, lower jaw 112 includes a first groove 194, and a second groove 195 formed on either side of a central slotted spline 196. Cartridge 122 is configured to be installed vertically into lower jaw 112 such that first and second side portions 122a, 122b fit within and are supported by first groove 194 and second groove 195, respectively, of lower jaw 112. When cartridge 122 is installed, slot 198 of central spline 196 is aligned with slot 199 of cartridge 122, allowing drive member 150 to translate distally through channel 129 upon actuation of the stapler and proximally after firing of staples 124. In preferred embodiments, lower jaw 112 may be formed of metals or metal alloys of sufficient mechanical strength to withstand the forces applied upon actuation of the stapler. Spline 196, slot 198, and slot 199, all extend in the direction of longitudinal axis X-X.

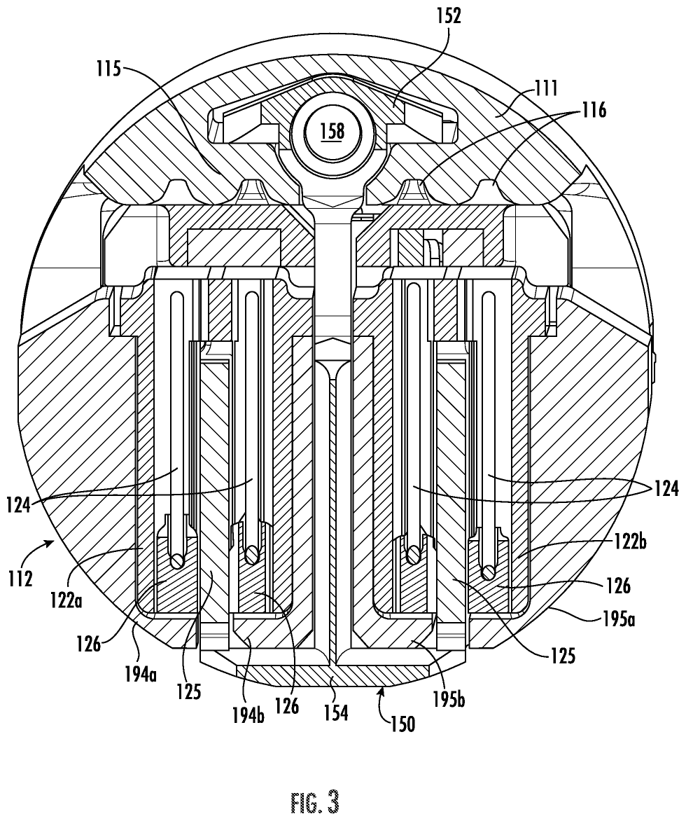

Referring now to FIG. 3, first and second grooves 194, 195 are configured to support both side portions 122a, 122b of cartridge 122. First groove 194 is on one side of spline 196 and includes outer wall 194a and inner wall 194b that support first side portion 122a of cartridge 122. Second groove 195 is on the opposite side of spline 196 and includes outer wall 195a and inner wall 195b that support second side portion 122b of cartridge 122. This configuration allows for both side portions 122a, 122b of plastic cartridge 122 to be reinforced on all lateral sides, inhibiting deformation of cartridge 122 in response to the forces applied when the stapler is actuated to fire staples through tissue grasped by jaws 111, 112. By inhibiting the deformation of side portions 122a, 122b of cartridge 122, the consistency of staple formation is improved.

Additionally, when cartridge 122 is installed, first and second grooves 194, 195 include central slots 197 (see FIG. 2B) that are substantially aligned with staple retention openings 127 of cartridge 122, allowing the inclined ramp portions 125 formed on drive member 150 to translate distally through central slots 197 upon actuation of the stapler and proximally upon retraction of drive member 150 after firing of staples 124.

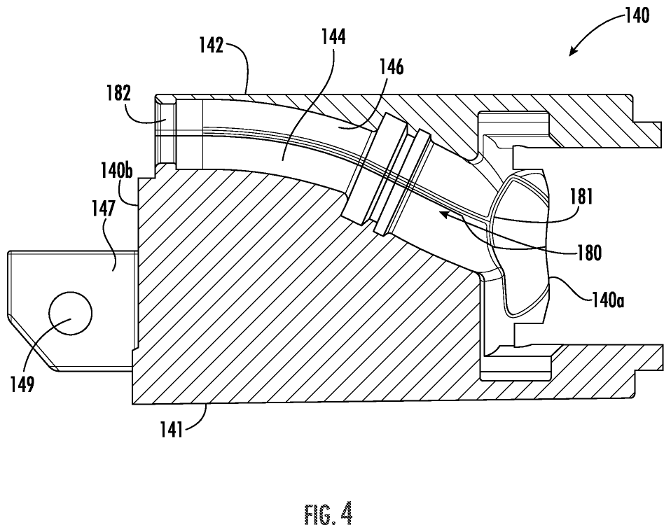

Referring now to FIG. 4, jaws 111, 112 are attached to surgical instrument 100 via a suitable coupling device, such as a clevis 140. Clevis 140 includes upper and lower portions that cooperate when assembled to form a protrusion 145 configured to engage tabs 113 (see FIG. 1A) of jaw 111 to securely mount jaw 111 in a fixed position on instrument 100. Clevis 140 further includes an opening for receiving a pivot pin 130 defining a pivot axis around which jaw 112 pivots as described in more detail below.

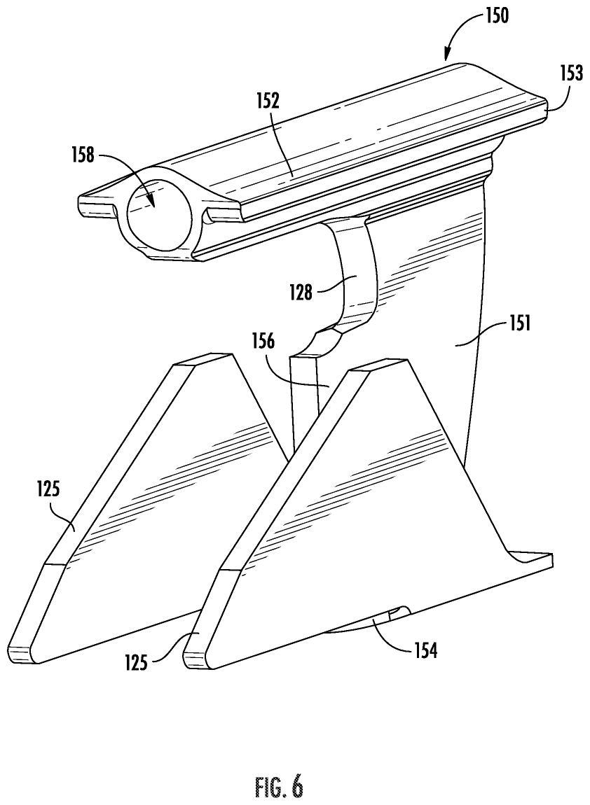

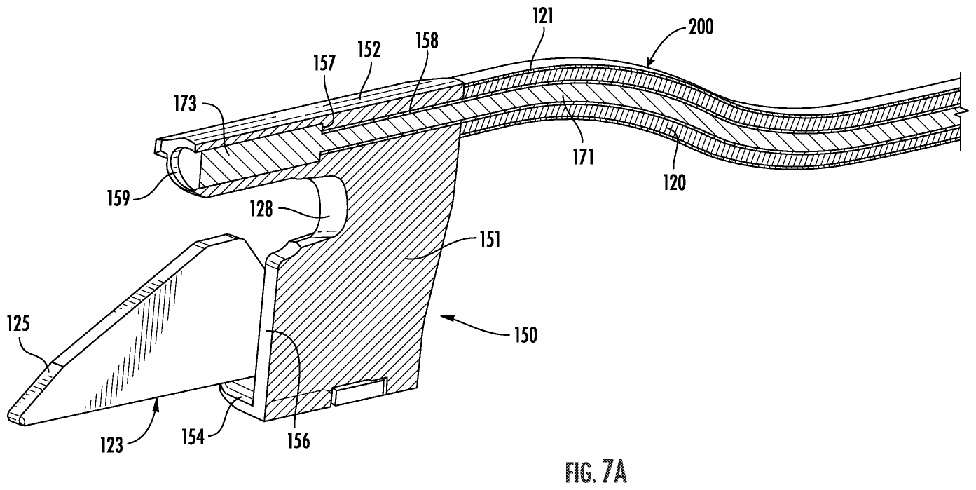

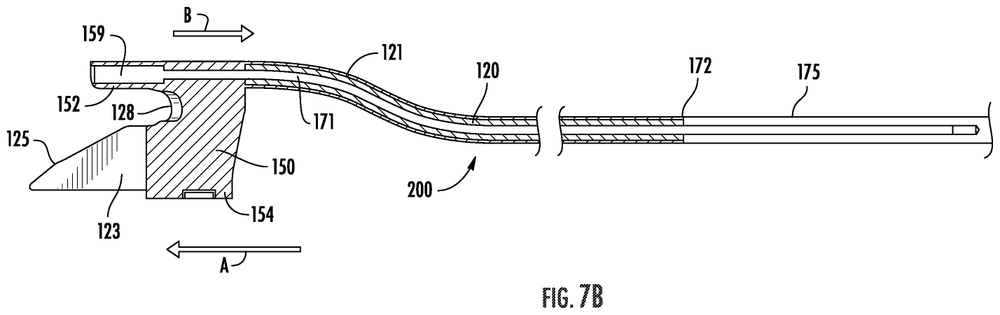

In an exemplary embodiment, clevis 140 includes a proximal surface 140a and a distal surface 140b. Clevis 140 further includes upper clevis portion 142 and lower clevis portion 141 that cooperate when assembled to form protrusion 145 (FIG. 1A) configured to engage tabs 113 of jaw 111 to securely mount jaw 111 in a fixed position on instrument 100. Lower clevis portion 141 includes a pair of distally extending arms 147 for supporting movable jaw 112. Arms 147 include opening 149 for receiving a pivot pin 130 defining a pivot axis around which jaw 112 pivots as described in more detail below. Lower clevis portion 141 also includes ramped groove 144 configured to guide a portion of an actuation coil 120 emerging from wrist 160. Upper clevis portion 142 includes a complementary shaped ramped groove 146 that cooperates with ramped groove 144 of lower clevis portion 141 to form an enclosed channel 180 that guides coil 120 as it jogs upwards from wrist 160 towards distal surface 157 of upper shoe 152 of drive member 150 (see FIG. 7). In certain embodiments, channel 180 may include a first end 181 at a central portion of proximal surface 140a and a second end 182 at a peripheral portion of distal surface 140b. In certain embodiments, enclosed channel 180 may be substantially “S” shaped. In other embodiments, channel 180 may be curved such that it has defines a concave surface facing lower clevis portion 141. Although shown as a two-part clevis, it should be understood that the clevis may be a unitary structure formed, for example, by molding, machining, 3-D printing, or the like.

The curved channel 180 of clevis 140 provides a passage for an actuation mechanism (discussed below) to advance or translate drive member 150 through, for example, the first jaw or anvil on the end effector. The channel 180 has a proximal end in the central portion of the surgical instrument for coupling with the drive assembly of the instrument and receiving the actuation mechanism, e.g., a coil or cable. The channel curves to a peripheral portion of the instrument to allow the actuation mechanism to pass through the first jaw of the end effector. This configuration allows the actuation mechanism to advance the drive member through the first jaw (instead of through the staple cartridge as in conventional surgical stapling instruments), thereby eliminating the internal channel for the actuation mechanism from the staple cartridge and providing more space in the cartridge for the staples and/or the reinforcing wall discussed above.

A more complete description of a suitable clevis 140 for use with the present invention may be found in commonly-assigned, co-pending provisional patent applications Ser. No. 62,783,444, 62,783,460, 62,747,912, and 62,783,429, the complete disclosures of which are hereby incorporated by reference in their entirety for all purposes. Of course, it will be recognized by those skilled in the art that other coupling mechanisms known by those skilled in the art may be used with the present invention to attach the jaws 111, 112 to the proximal portion of surgical instrument 100.

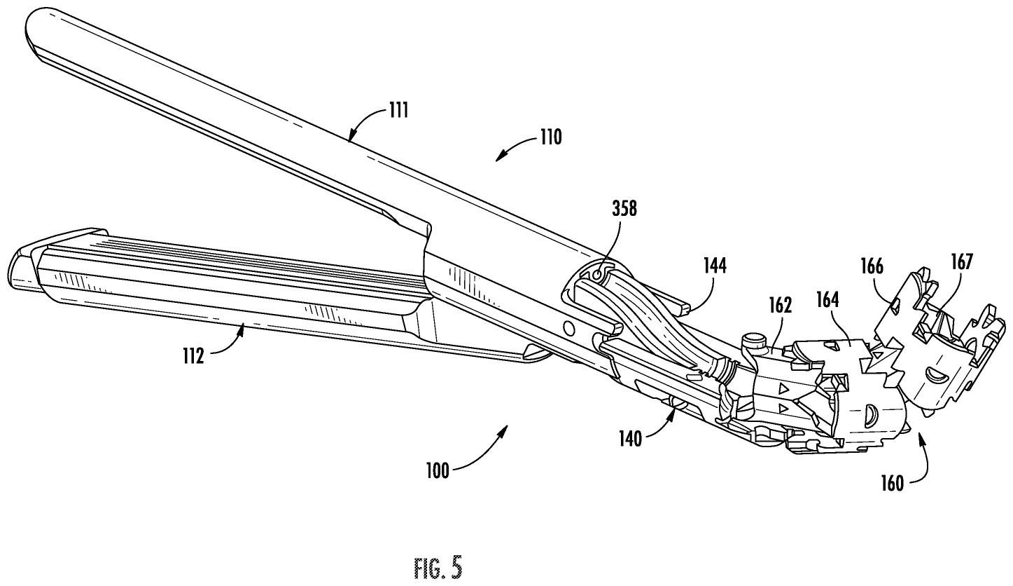

Referring now to FIG. 5, end effector 110 may be articulated in multiple directions by an articulation mechanism. In certain embodiments, the articulation mechanism may be a wrist assembly 160 as shown, although other articulation mechanisms are contemplated. In an exemplary embodiment, wrist 160 includes a plurality of articulation joints 162, 164, 166, etc. that define a bore 167 through which an actuation mechanism (in embodiments, coil 120 and drive cable 171, see FIG. 7A) may pass. Upon exiting articulation wrist 160, coil 120 enters and passes through channel 180 of clevis 140, ultimately engaging proximal surface 153 of upper shoe 152 of drive member 150. Other articulation mechanisms are known by those skilled in the art that may substitute for wrist 160; See for example U.S. Publication. No. 2015/0250530 and U.S. Provisional Application Ser. No. 62/783,481, filed Dec. 21, 2018, the complete disclosures of which are hereby incorporated by reference in their entirety.

As shown in FIG. 6, an illustrative drive member 150 may include a body 151, an upper shoe 152, a lower shoe 154, a central portion 156 and a pair of inclined distal surfaces or ramps 125 spaced laterally outward from central portion 156. In other embodiments, drive member 150 may include one inclined ramp portion or more than two inclined ramp portions (depending on the number of rows of staples 124 and pushers 126 within staple cartridge 122). Ramps 125 are preferably integral with drive member 150. In conventional stapling instruments, the shuttle 123 (which includes ramps 125) is typically formed as part of the lower jaw or within the staple cartridge. In the present disclosure, shuttle 123 and ramps 125 are formed as part of the drive member 150. This design allows for a more compact and efficient end effector for the surgical instrument and provides more space in the lower jaw for reinforcing the staple cartridge, as discussed above.

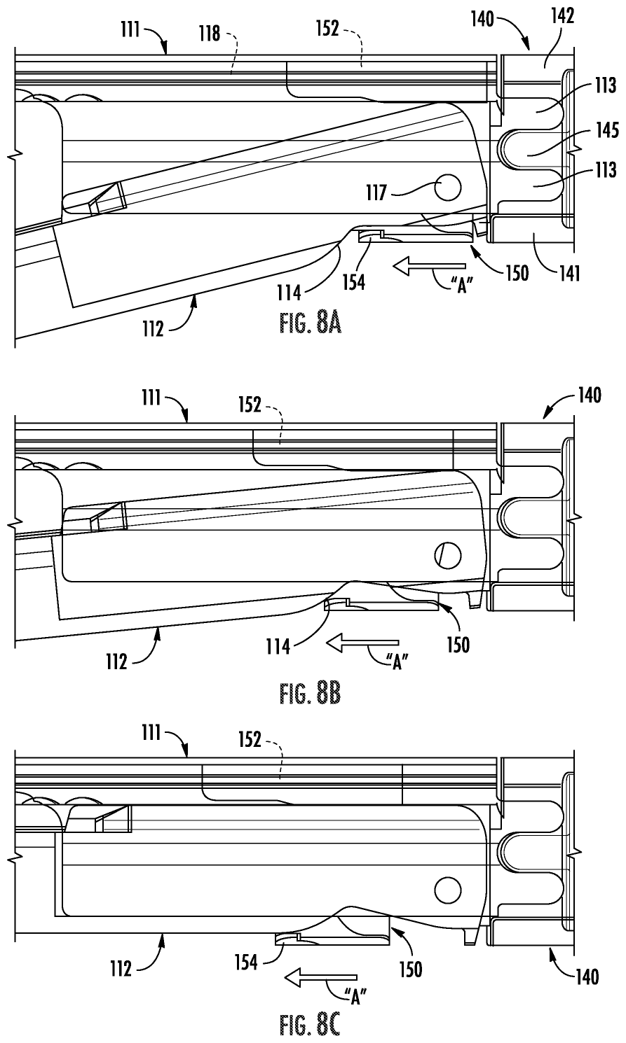

Upper shoe 152 of drive member 150 is substantially aligned with and translates through a channel 118 (see FIG. 8A) in fixed jaw 111, while lower shoe 154 of drive member 150 is substantially aligned with and translates through a channel (not shown) and below jaw 112. Bore 158 is formed through upper shoe 152 to receive drive cable 171 as will be described in more detail below. Proximal surface 153 of upper shoe 152 is configured to be engaged by a coil 120 of actuation assembly 200 such that coil 120 may apply force to upper shoe 152 to advance drive member 150 distally, i.e., in the direction of arrow “A” in FIG. 7B. In certain embodiments, drive member 150 may also include a knife 128 configured to translate distally through a channel 114 in cartridge 122 and to sever clamped, stapled tissue. In certain embodiments, knife 128 may simply be a sharpened edge on drive member 150 rather than a distinct structure within the cartridge.

Referring now to FIGS. 7A and 7B, actuation assembly 200 includes a drive cable 171, a coil 120, a sheath 121 surrounding coil 120, and a proximal drive rod 175. Drive cable 171 includes an enlarged distal end 173. Upper shoe 152 of drive member 150 includes bore 158 into which drive cables 171 are routed. When assembling illustrative surgical instrument 100, coil 120 and a protective sheath 121 are slipped over the free end of drive cable 171. The free end of drive cable 171 is attached to a drive rod 175 securing coil 120 and the protective sheath 121 between drive member 150 and drive rod 175 as seen in FIG. 7B. Sheath 121 may function to promote stability, smooth movement, and prevent buckling upon actuation of surgical instrument 100. Sheath 121 may be made from polyimide, or any other suitable material having the requisite strength requirements such as various reinforced plastics, a nickel titanium alloy such as NITINOL™, poly para-phenyleneterphtalamide materials such as KEVLAR™ commercially available from DuPont. Those of skill in the art may envision other suitable materials.

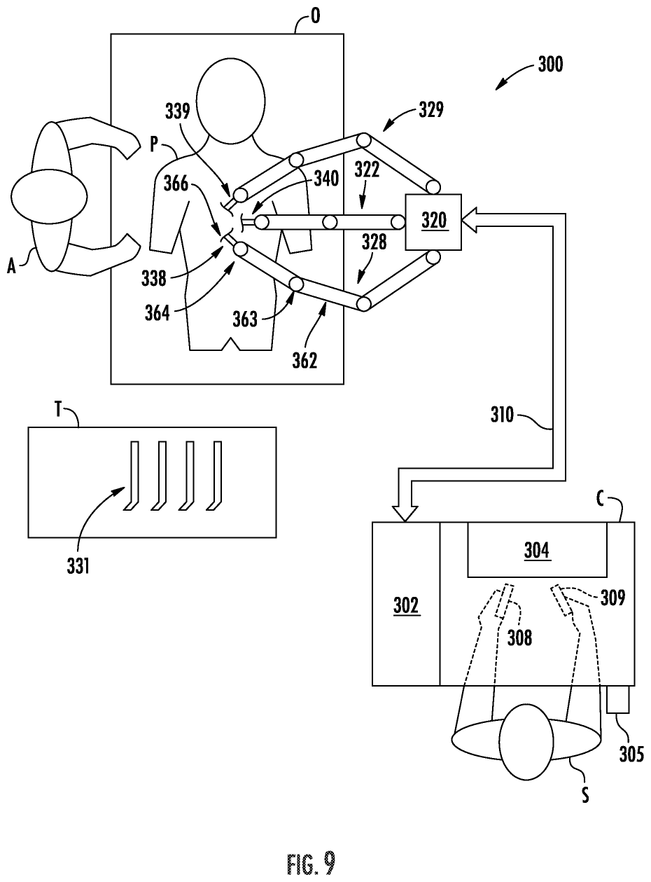

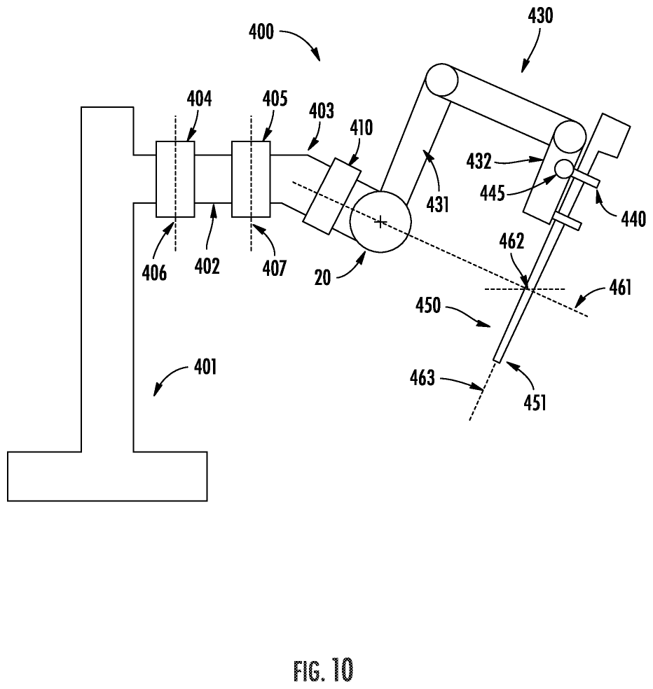

The proximal surface of upper shoe 152 is configured to be engaged by a coil 120 of actuation assembly 200 such that coil 120 may apply force to upper shoe 152 to advance drive member 150 distally. A knife 128 may be formed on drive member 150 along the distal edge between upper shoe 152 and central portion 156. Enlarged distal end 173 of drive cable 171 resides within an enlarged distal portion 159 of bore 158 in upper shoe 152 of drive member 150, such that the proximal face 157 of enlarged distal end 173 may apply a retraction force on upper shoe 152 when the drive cable 171 is pulled proximally, i.e., in the direction of arrow “B” in FIG. 7B. The drive rod is operationally connected to an actuator (e.g., input couplers 120), which allows distal translation and proximal retraction of actuation assembly 190. Those skilled in the art will recognize that in a manually actuated instrument, the actuator may be a movable handle, such as moveable handle 102b shown in FIG. 1; in a powered instrument the actuator may be a button (not shown) that causes a motor to act on the drive rod; and in a robotic system, the actuator may be a control device such as the control devices described below in connection with FIGS. 9 and 10. Any suitable backend actuation mechanism for driving the components of the surgical stapling instrument may be used. For additional details relating to exemplary actuation mechanisms using push/pull drive cables see, e.g., commonly owned International Application WO 2018/049217, the disclosure of which is hereby incorporated by reference in its entirety.

During actuation of illustrative surgical instrument 100, drive rod 175 applies force to coil 120, thereby causing coil 120 to apply force to upper shoe 152 of drive member 150, translating it distally (i.e., in the direction of arrow “A” in FIG. 7B) initially closing jaws 111,112 and then ejecting staples 124 from cartridge 122 to staple tissue. After stapling is complete, drive rod 175 applies a force in the proximal direction to effect retraction of drive member. During retraction, enlarged distal end 173 of drive cable 171 is obstructed by wall 157 of enlarged portion 159 of bore 158, causing drive cable 171 to apply force to upper shoe 152 of drive member 150, thereby translating drive member 150 in the proximal direction. In certain embodiments, the surgical instrument may be designed such that the drive member 150 is not retracted in the proximal direction after the staples have been fired. One of ordinary skill in the art will appreciate that drive member 150, drive cable 171, and drive rod 175 all move in unison and remain in the same relative position to each other.

Upon actuation of the surgical instrument, drive member 150 is advanced distally through end effector 110 to move jaws 111, 112 from the open position to the closed position, after which shuttle 123 and knife 128 are advanced distally through a staple cartridge to staple and cut tissue grasped between jaws 111, 112. Of course, it will be recognized by those skilled in the art that drive member 150 may be any structure capable of pushing at least one of a shuttle or a knife of a surgical stapling instrument with the necessary force to effectively sever or staple human tissue. Drive member 150 may be an I-beam, an E-beam, or any other type of drive member capable of performing similar functions. Drive member 150 is movably supported on the surgical stapling instrument 100 such that it may pass distally through a staple cartridge and upper fixed jaw 111 and lower jaw 112 when the surgical stapling instrument is fired (e.g., actuated).

In the preferred embodiment, drive cable 171 advances drive member 150 through fixed jaw 111 (instead of through the staple cartridge jaw as in conventional surgical stapling instruments). Eliminating the internal channel for the actuation mechanism from the staple cartridge provides more space in the cartridge for the staples and for the reinforcing wall discussed above. In alternative embodiments, coil 120 of actuation assembly 190 may be coupled with lower shoe 154 instead of upper shoe 152. In these embodiments, coil 120 applies force to lower shoe 153 to advance drive member 150 distally through a channel (not shown) in the lower jaw 112. In these embodiments, coil 120 will advance at least through a portion of lower jaw 112 and staple cartridge 122.

FIGS. 8A-C depict fixed jaw 111 and movable jaw 112 of illustrative surgical instrument 100 sequentially moving from an open configuration to a closed configuration. As shown in FIG. 8A, in the open configuration, drive member 150 is positioned proximally of cam surface 114 formed on movable jaw 112. As drive member 150 translates in the distal direction “A” movable jaw 112 will rotate towards the closed position around pivot 117.

In FIG. 8B, drive member 150 has come into contact with cam surface 114 of movable jaw 112. As lower portion 154 of drive member 150 rides underneath cam surface 114, drive member 150 pushes movable jaw 112, causing it to pivot towards the closed position.

FIG. 8C illustrates jaws 111, 112 in the closed position. Drive member 150 has translated distally past cam surface 114. In this position, tissue is clamped, and further advancement of the drive member will sever and staple tissue.

FIG. 9 illustrates, as an example, a top view of an operating room employing a robotic surgical system. The robotic surgical system in this case is a robotic surgical system 300 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The Console includes a monitor 304 for displaying an image of a surgical site to the Surgeon, left and right manipulatable control devices 308 and 309, a foot pedal 305, and a processor 302. The control devices 308 and 309 may include any one or more of a variety of input devices such as joysticks, gloves, trigger-guns, hand-operated controllers, or the like. The processor 302 may be a dedicated computer that may be integrated into the Console or positioned next to it.

The Surgeon performs a minimally invasive surgical procedure by manipulating the control devices 308 and 309 (also referred to herein as “master manipulators”) so that the processor 302 causes their respectively associated robotic arm assemblies, 328 and 329, (also referred to herein as “slave manipulators”) to manipulate their respective removably coupled surgical instruments 338 and 339 (also referred to herein as “tools”) accordingly, while the Surgeon views the surgical site in 3-D on the Console monitor 304 as it is captured by a stereoscopic endoscope 340.

Each of the tools 338 and 339, as well as the endoscope 340, may be inserted through a cannula or other tool guide (not shown) into the Patient so as to extend down to the surgical site through a corresponding minimally invasive incision such as incision 366. Each of the robotic arms is conventionally formed of links, such as link 362, which are coupled together and manipulated through motor controlled or active joints, such as joint 363.

The number of surgical tools used at one time and consequently, the number of robotic arms being used in the system 300 will generally depend on the diagnostic or surgical procedure and the space constraints within the operating room, among other factors. If it is necessary to change one or more of the tools being used during a procedure, the Assistant may remove the tool no longer being used from its robotic arm, and replace it with another tool 331 from a Tray (“T”) in the operating room.

The monitor 304 may be positioned near the Surgeon's hands so that it will display a projected image that is oriented so that the Surgeon feels that he or she is actually looking directly down onto the operating site. To that end, images of the tools 338 and 339 may appear to be located substantially where the Surgeon's hands are located.

The processor 302 performs various functions in the system 300. One important function that it performs is to translate and transfer the mechanical motion of control devices 308 and 309 to their respective robotic arms 328 and 329 through control signals over bus 310 so that the Surgeon can effectively manipulate their respective tools 338 and 339. Another important function is to implement various control system processes as described herein.

Although described as a processor, it is to be appreciated that the processor 302 may be implemented in practice by any combination of hardware, software and firmware. Also, its functions as described herein may be performed by one unit, or divided up among different components, each of which may be implemented in turn by any combination of hardware, software and firmware. For additional details on robotic surgical systems, see, e.g., commonly owned U.S. Pat. No. 6,493,608 “Aspects of a Control System of a Minimally Invasive Surgical Apparatus,” and commonly owned U.S. Pat. No. 6,671,581 “Camera Referenced Control in a Minimally Invasive Surgical Apparatus,” which are hereby incorporated herein by reference in their entirety for all purposes.

FIG. 10 illustrates, as an example, a side view of a simplified (not necessarily in proportion or complete) illustrative robotic arm assembly 400 (which is representative of robotic arm assemblies 328 and 329) holding a surgical instrument 450 (which is representative of tools 338 and 339) for performing a surgical procedure. The surgical instrument 450 is removably held in tool holder 440. The arm assembly 400 is mechanically supported by a base 401, which may be part of a patient-side movable cart or affixed to the operating table or ceiling. It includes links 402 and 403, which are coupled together and to the base 401 through setup joints 404 and 405.

The setup joints 404 and 405 in this example are passive joints that allow manual positioning of the arm 400 when their brakes are released. For example, setup joint 404 allows link 402 to be manually rotated about axis 406, and setup joint 405 allows link 403 to be manually rotated about axis 407. Although only two links and two setup joints are shown in this example, more or less of each may be used as appropriate in this and other robotic arm assemblies in conjunction with the present invention. For example, although setup joints 404 and 405 are useful for horizontal positioning of the arm 400, additional setup joints may be included and useful for limited vertical and angular positioning of the arm 400. For major vertical positioning of the arm 400, however, the arm 400 may also be slidably moved along the vertical axis of the base 401 and locked in position.

The robotic arm assembly 400 also includes three active joints driven by motors. A yaw joint 410 allows arm section 430 to rotate around an axis 461, and a pitch joint 420 allows arm section 430 to rotate about an axis perpendicular to that of axis 461 and orthogonal to the plane of the drawing. The arm section 430 is configured so that sections 431 and 432 are always parallel to each other as the pitch joint 420 is rotated by its motor. As a consequence, the instrument 450 may be controllably moved by driving the yaw and pitch motors so as to pivot about the pivot point 462, which is generally located through manual positioning of the setup joints 404 and 405 so as to be at the point of incision into the patient. In addition, an insertion gear 445 may be coupled to a linear drive mechanism (not shown) to extend or retract the instrument 450 along its axis 463.

Although each of the yaw, pitch and insertion joints or gears, 410, 420 and 445, is controlled by an individual joint or gear controller, the three controllers are controlled by a common master/slave control system so that the robotic arm assembly 400 (also referred to herein as a “slave manipulator”) may be controlled through user (e.g., surgeon) manipulation of its associated master manipulator. A more complete description of illustrative robotic surgical systems for use with the present invention can be found in commonly-assigned U.S. Pat. Nos. 9,295,524, 9,339,344, 9,358,074, and 9,452,019, the complete disclosures of which are hereby incorporated by reference in their entirety for all purposes.

While several embodiments have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus, the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present disclosure is intended to embrace all such alternatives, modifications and variances. As well, one skilled in the art will appreciate further features and advantages of the present disclosure based on the above-described embodiments. Accordingly, the present disclosure is not to be limited by what has been particularly shown and described, except as indicated by the appended claims.

1. A surgical instrument comprising:
an end effector comprising first and second jaws configured to move relative to each other from an open position to a closed position;
a staple cartridge removably coupled to the second jaw, wherein the second jaw has a cavity for receiving the staple cartridge, and at least one reinforcing wall extending through the cavity to provide support for the staple cartridge; and
a drive member configured to translate distally through the end effector to engage the staple cartridge.

2. The surgical instrument of claim 1, wherein the cavity comprises first and second outer walls and a distal wall and are configured to receive the staple cartridge from a direction lateral to the longitudinal axis of the surgical instrument.

3. The surgical instrument of claim 1, wherein the reinforcing wall defines first and second longitudinal grooves within the cavity of the second jaw.

4. The surgical instrument of claim 3, wherein the staple cartridge comprises first and second portions configured for positioning within the first and second grooves of the cavity of the second jaw.

5. The surgical instrument of claim 4, wherein the reinforcing wall comprises first and second walls defining a channel therebetween.

6. The surgical instrument of claim 5, wherein the drive member comprises a first portion. configured to translate through a channel in the first jaw, the instrument further comprising an actuation mechanism configured to translate the first portion of the drive member through the channel.

7. The surgical instrument of claim 6, wherein the drive member comprises a second portion configured to translate through the channel between the first and second walls of the reinforcing wall.

8. The surgical instrument of claim 1, wherein each of the first and second portions of the staple cartridge contains at least one row of staple retention openings.

9. The surgical instrument of claim 3, wherein the second jaw comprises a slotted spine between the first and second grooves, the slotted spine having a longitudinal channel.

10. The surgical instrument according to claim 1, wherein the staple cartridge comprises a polymer material and at least a portion of the second jaw is metal.

11. The surgical instrument according to claim 6, further comprising an actuator for actuating the actuation mechanism, wherein the actuator includes a control device of a robotic surgical system.

12. The surgical instrument of claim 1, wherein the staple cartridge comprises a plurality of staples and the drive member comprise one or more inclined ramps configured to engage the staples and drive the staples in a direction transverse to the longitudinal axis as the drive member is translated longitudinally through the housing.

13. The surgical instrument of claim 12, wherein the one or more inclined ramps are integral with the drive member.

14. A staple cartridge for a surgical stapling instrument, the cartridge comprising:
an elongate base configured for placement into a cavity of a jaw of the surgical stapling instrument;
first and second side portions extending from the base in a direction substantially perpendicular to the longitudinal axis of the base and defining a substantially longitudinal channel therebetween; and
wherein the first and second side portions each include at least one row of staple retention openings.

15. The staple cartridge of claim 14, wherein the staple cartridge comprises a polymer material.

16. The staple cartridge of claim 14, wherein each opening in at least one row of staple retention openings contain a staple on a staple pusher.

17. The staple cartridge of claim 14, wherein the first and second side portions each include two rows of staple retention openings.

18. The staple cartridge of claim 14, wherein the first and second side portions are configured to receive at least one reinforcing wall extending through the cavity of the jaw of the surgical stapling instrument.

19. The staple cartridge of claim 14, wherein the staple cartridge is configured for placement into the jaw from a direction lateral to the longitudinal axis of the surgical instrument.

20. The staple cartridge of claim 18, wherein the reinforcing wall defines first and second longitudinal grooves within the cavity of the second jaw and the staple cartridge comprises first and second portions configured for positioning within the first and second longitudinal grooves.
